(19) United States
(12) Patent Application Publication
(10) Pub. No.: US 2018/0169362 A1
(43) Pub. Date: Jun. 21, 2018

(54) TRACHEOSTOMY TUBE ASSEMBLIES AND INNER CANNULAE

(71) Applicant: SMITHS MEDICAL INTERNATIONAL LIMITED, Ashford (GB)

(72) Inventors: Timothy Bateman, Hythe (GB); Stephen James Field, Canterbury (GB); Andrew Thomas Jeffrey, Hythe (GB); Neil Steven Veasey, Ashford (GB); Christopher John Woosnam, Great Sutton (GB)

(73) Assignee: SMITHS MEDICAL INTERNATIONAL LIMITED, Ashford (GB)

(21) Appl. No.: 15/736,449

(22) PCT Filed: Jun. 15, 2016
(86) PCT No.: PCT/GB2016/000120

§ 371 Date: Dec. 14, 2017

(30) Foreign Application Priority Data
Jul. 15, 2015 (GB) 1512674.1

(51) Int. Cl. A61M 16/04

(52) U.S. Cl. CPC A61M 16/0427; A61M 16/0465

(57) ABSTRACT

An inner cannula (2) for a tracheostomy tube (1) has a resilient catch (23) at its machine end. The catch has a two angular teeth (27) and (28) projecting outwardly towards its free end. One tooth (27) is shaped and positioned to engage in a groove (40) on the inside of a hub (16) at the machine end of the tube when the inner cannula is fully inserted. The other tooth (28) is smaller and is spaced rearwardly along the catch. The hub (16) also has a shallow rounded bead (44) projecting inwardly and spaced a short distance to the rear of the groove (40). When the inner cannula (2) is pulled out of the tube (1) the smaller tooth (28) engages the bead (44) on the hub (16) causing the catch (23) to deflect inwardly. In this way, the larger tooth (27) is displaced slightly out of the groove (40) making it easier for it to ramp out of the groove and allow the cannula to be withdrawn with little additional pulling force.

This invention relates to tracheostomy tube assemblies of the kind of a tracheostomy tube and an inner cannula extending within the tracheostomy tube, the inner cannula including a tubular shaft and a machine end region arranged to fit within a hub at the machine end of the tracheostomy tube.

Tracheostomy tube assemblies commonly include an outer tube and an inner tube or cannula that is a removable fit within the outer tube. The inner cannula can be removed and replaced periodically to ensure that the passage through the assembly does not become blocked by secretions. This avoids the need to remove the outer tube frequently.

The inner cannula presents various problems because it must be thin walled and a close fit within the outer tube so as to provide a large bore and thereby limit the resistance to flow of gas along the assembly. It must, however, also be sufficiently stiff to be inserted in the outer tube without buckling or kinking and must be readily removable, preferably with only minimal force being exerted on the tube. WO94/01156 and WO2004/101048 describe inner cannulae made of PTFE. EP1938857 describes an arrangement of tracheostomy tubes and inner cannulae where the hubs of the inner cannulae of different sizes are shaped differently so that they will only fit in the appropriate tracheostomy tube. EP2224985 describes an arrangement for attaching a hub to the shaft of an inner cannula. GB2056285 describes an inner cannula having a wall corrugated both externally and internally and a longitudinal groove or other reinforcement member traversing at least some of the corrugations. U.S. Pat. No. 4,817,598 describes a smooth-walled inner cannula having a ring-pull formation at its rear, machine end. U.S. Pat. No. 5,119,811 describes an inner cannula with a flared patient end and formed of two layers of different materials. U.S. Pat. No. 5,386,826 describes an inner cannula with an outer helical filament or layer of low friction material. U.S. Pat. No. 5,983,895 describes an inner cannula with straight sections at opposite ends joined by an intermediate curved section. U.S. Pat. No. 6,019,753 describes an inner cannula with two elongate regions of different flexibility so that the cannula has a plane of preferential bending. U.S. Pat. No. 6,019,753 describes an inner cannula having a shaft formed with slots to make it more flexible, the slots being covered by an outer thin sheath. U.S. Pat. No. 6,135,110 describes a curved inner cannula that is retained with the outer tube by means of a rotatable spring fitting.

It is an object of the present invention to provide an alternative inner cannula and tracheostomy tube assembly.

According to one aspect of the present invention there is provided an assembly of the above-specified kind, characterised in that the machine end region of the inner cannula has a resilient catch arranged to engage the inside of the hub at the machine end of the tracheostomy tube to retain the inner cannula in the hub of the tracheostomy tube against an axial force tending to withdraw the inner cannula, that the catch has a first outwardly-directed surface formation and a second outwardly-directed surface formation located closer the machine end of the inner cannula, that the inner surface of the hub has a first surface formation and a second surface formation located closer the machine end of the tracheostomy tube, that the surface formations on the inner cannula and tracheostomy tube are arranged such that when force is applied to the inner cannula to withdraw it from the tracheostomy tube the second formation on the inner cannula engages the second formation on the hub before the first formation on the inner cannula engages the first formation on the hub, such that engagement of the second formations acts to displace the catch inwardly so as to facilitate passage of the first formation on the inner cannula over the first formation on the hub during withdrawal of the inner cannula from the tracheostomy tube.

The first and second surface formations on the inner cannula preferably both have an inclined surface facing towards the machine end of the cannula. The surface formations on the catch preferably project radially outwardly, the first surface formation on the hub preferably being a groove. The first surface formation on the catch and the first surface formation on the hub are preferably triangular. The second surface formation on the hub may be a rounded formation projecting inwardly of the hub. The second formation on the catch preferably has a lower height than the first formation on the catch. The second formation on the catch is preferably triangular with a forward face that projects substantially vertically of the catch and a rear face that is inclined. The catch may be formed from a wall of an end fitting at the machine end of the inner cannula. The catch may be a substantially rectangular.

According to another aspect of the present invention there is provided an assembly of a tracheostomy tube and an inner cannula extending within the tracheostomy tube, the inner cannula including a tubular shaft and a machine end region arranged to fit within a hub at the machine end of the tracheostomy tube, characterised in that the machine end region of the inner cannula has a resilient catch in the machine end region arranged to engage the inside of the hub at the machine end of the tracheostomy tube to retain the inner cannula in the tracheostomy tube against an axial force tending to withdraw the inner cannula, that the catch has two inclined projections spaced from one another along it, and that the inner surface of the hub has a recess shaped to receive one of the projections on the catch and a projection positioned to engage the other projection on the catch and thereby deflect the catch inwardly when the inner cannula is pulled rearwardly out of the tube.

According to a further aspect of the present invention there is provided an inner cannula for an assembly according to the above one or other aspect of the present invention.

A tracheostomy tube assembly including an inner cannula both in accordance with the present invention, will now be described, by way of example, with reference to the accompanying drawings, in which:

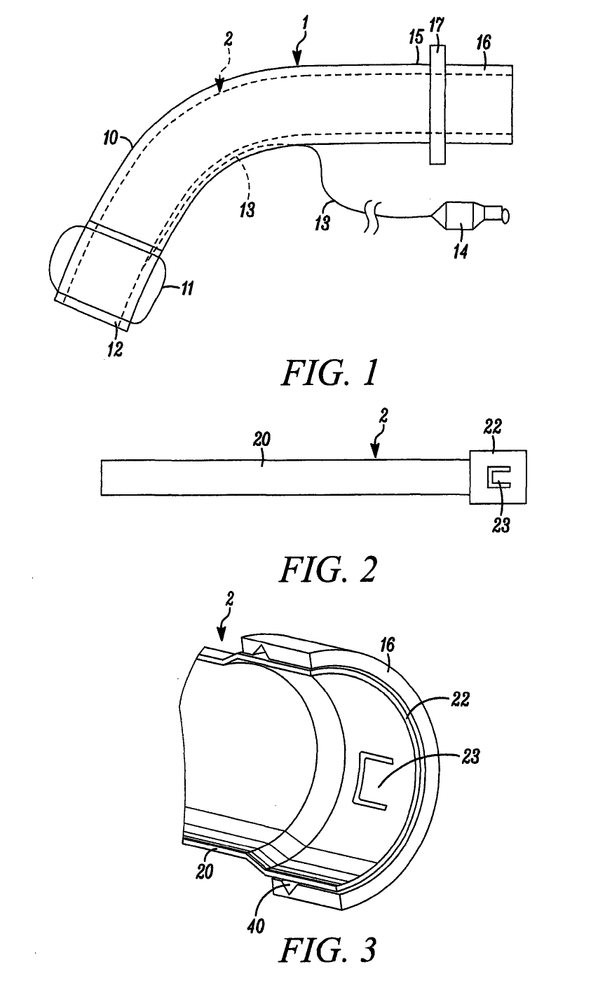

FIG. 1 is a side elevation view of a tracheostomy tube assembly including an inner cannula;

FIG. 2 is a side elevation view of the inner cannula;

FIG. 3 is an enlarged perspective, cutaway view of the inside of the machine end of the inner cannula and the hub of the tracheostomy tube;

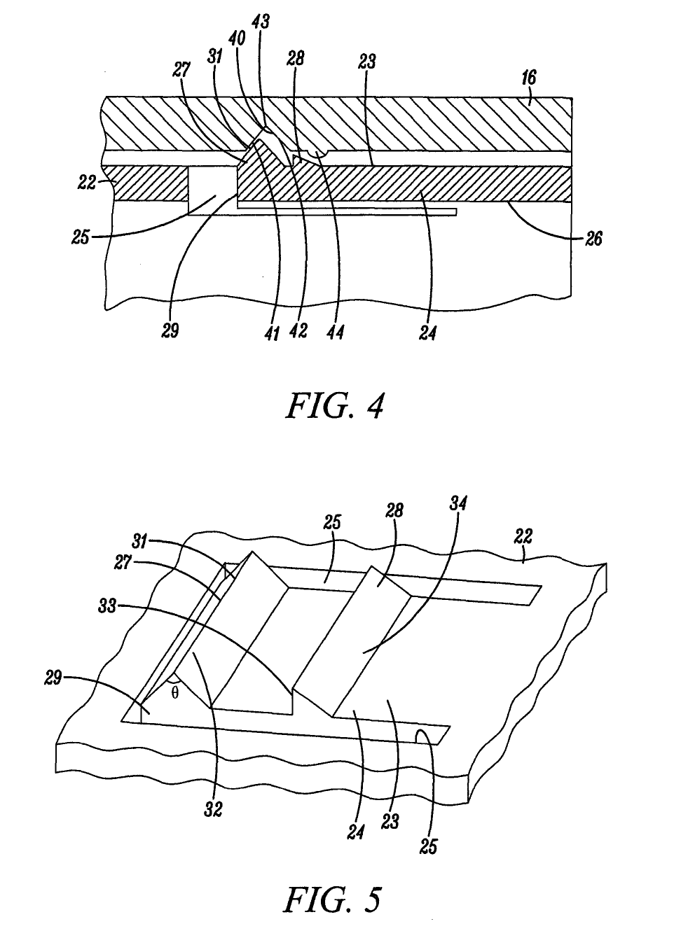

FIG. 4 is a cross-sectional, side elevation view of the machine end of the inner cannula engaged within the hub of the tracheostomy tube; and

FIG. 5 is a perspective view of a catch on the inner cannula.

With reference first to FIGS. 1 and 2, the tracheostomy tube assembly comprises an outer tracheostomy tube 1 and a removable inner cannula 2 inserted within the outer tube. The outer tube 1 has a shaft 10 with an inflatable sealing cuff 11 embracing the shaft close to the patient end 12 of the tube, the cuff being inflated via an inflation lumen 13 and a combined connector and inflation indicator 14. At its rear or machine end 15 the outer tube 1 has a hub 16 and flange 17 to which a retaining tape (not shown) can be fastened for securing the tube with the patient's neck. The outer tube 1 could have an internal diameter between about 2 mm and 10 mm, and its length could be between 60 mm and 200 mm.

The inner cannula 2 includes a shaft 20 of circular section having at its rear or machine end an integrally moulded machine end fitting 22. The shaft 20 is of a thin, stiff plastics material, such as PVC, polyurethane, polyethylene, polypropylene, PTFE or other flexible or semi-rigid plastics material. The external diameter of the shaft 20 is selected to be just smaller than the inner diameter of the shaft 10 of the outer tube 1 so that the inner cannula 2 can be readily inserted and removed from the outer tube. The machine end fitting 22 is shaped to locate within the hub 16 of the tracheostomy tube 1 and prevent the inner cannula 2 extending beyond the patient end 12 of the tube. The end fitting 22 is also arranged to be retained securely within the hub 16 until the inner cannula needs to be removed. This is achieved by means of cooperating surface formations on the machine end fitting 22 of the inner cannula 2 and on the inside of the hub 16 of the tracheostomy tube 1.

The surface formations on the inner cannula 2 are provided by one or more (typically two or three) resilient catches 23 as shown most clearly in FIGS. 4 and 5 projecting outwardly around the outside of the end fitting 22. The or each catch 23 includes a rectangular tab 24 formed from or otherwise attached with the wall of the machine end fitting 22 at its machine end only and separated around its sides and patient end by a U-shape gap 25 so that the patient end 29 of the tab is free for resilient displacement inwardly or outwardly. The inner surface 26 of the catch 23 is plain, following the shape of the remainder of the end fitting 22. The outer surface, however, is profiled to provide the surface formations mentioned above. More particularly, the outer surface is formed with two surface formations 27 and 28 spaced from one another along the length of the catch 23. The first surface formation 27 is located at the free, patient end 29 of the catch 23 and takes the form of a lateral tooth of isosceles triangular section with two inclined faces 31 and 32 having an included angle θ of 90°. The height of the first tooth 27 is about equal to the wall thickness of the end fitting 22. The second surface formation 28 is spaced a short distance rearwardly (towards the machine end) and is in the form of a tooth but has a vertical forward face 33 and an inclined rear face 34 that slopes at about 30° to the axis of the assembly. The second tooth 28 is only about half the height of the first tooth 27.

The surface formations of the inside of the hub 16 of the tracheostomy tube 1 are arranged to cooperate with the or each catch 23 on the inner cannula 2. As shown in FIG. 4, the inside surface of the hub 16 is moulded with an annular groove 40 extending around the hub at a position where it aligns with the first tooth 27 on the or each catch 23. The groove 40 has a triangular section of substantially the same size and shape as the first tooth 27, with two walls 41 and 42 inclined at 90° and an apex 43 that is slightly rounded. Although the groove 40 has substantially the same shape as the tooth 27, the tooth does not fill the groove because there is a small spacing between the outside of the end fitting 22 and the inside of the hub 16, which means that the tooth projects into the groove by about half the depth of the groove. The inside of the hub 16 also has a further surface formation in the form of a projecting rounded annular bead 44 of semicircular section located a short distance rearwardly of the groove 40. The bead 44 is arranged to contact the second tooth 28 when the inner cannula 2 is pulled rearwardly out of the tracheostomy tube 1 to a position where the first tooth 27 is aligned approximately centrally in the groove 40. In this position the bead 44 engages the sloping rear face 34 of the second tooth 28 about half way along its length. Pulling the inner cannula 2 further out of the tracheostomy tube 1 causes the tab 24 of the catch 23 to be resiliently deflected inwardly as the rear face of the 34 of the second tooth 28 slides along the bead 44. The relatively small deflection of the tab 24 at the second tooth 28 causes a magnified deflection of the free, patient end 29 of the tab and thereby displaces the first tooth 27 by a relatively large amount sufficient to move its apex to a position close to the edge of the groove 40, that is, a position close to the plane of the cylindrical inner surface of the hub 16. In this position relatively little additional force is needed to ride the apex of the first tooth 27 fully out of the groove 40 and thereby enable the inner cannula 2 to be pulled completely out of the outer tracheostomy tube 1.

A replacement inner cannula can be easily inserted since the forward end of the first tooth 27 is deformed inwardly when its inclined surface 31 engages the opening of the hub 16 on the outer tube. The tooth 27 rides over the bead 44 and then snaps into the groove 40 in the hub 16 when the inner cannula 2 has been fully inserted.

The machine end of the inner cannula 2 could include a hinged ring-pull moulding or some other arrangement to provide a member that can be gripped when the inner cannula needs to be pulled out of the outer tube.

The arrangement of the present invention therefore enables an inner cannula to be retained securely within the outer tracheostomy tube whilst also enabling the inner cannula to be pulled out of the tube with less force than would be needed with a simple catch. This helps overcome the problem with some previous assemblies where a secure retention of the inner cannula can only be achieved in assemblies where a relatively high force is needed to remove and replace the inner cannula.

1-11. (canceled)

12. An assembly of a tracheostomy tube and an inner cannula extending within the tracheostomy tube, the inner cannula including a tubular shaft and a machine end region arranged to fit within a hub at the machine end of the tracheostomy tube, characterised in that the machine end region of the inner cannula has a resilient catch arranged to engage the inside of the hub at the machine end of the tracheostomy tube to retain the inner cannula in the hub of the tracheostomy tube against an axial force tending to withdraw the inner cannula, that the catch has a first outwardly-directed surface formation and a second outwardly-directed surface formation located closer to the machine end of the inner cannula, that the inner surface of the hub has a first surface formation and a second surface formation located closer the machine end of the tracheostomy tube, that the surface formations on the inner cannula and tracheostomy tube are arranged such that when force is applied to the inner cannula to withdraw it from the tracheostomy tube the second formation on the inner cannula engages the second formation on the hub before the first formation on the inner cannula engages the first formation on the hub, such that engagement of the second formations acts to displace the catch inwardly so as to facilitate passage of the first formation on the inner cannula over the first formation on the hub during withdrawal of the inner cannula from the tracheostomy tube.

13. An assembly according to claim 12, characterised in that the first and second surface formations on the inner cannula both have an inclined surface facing towards the machine end of the cannula.

14. An assembly according to claim 12, characterised in that the surface formations on the catch project radially outwardly, and that the first surface formation on the hub is a groove.

15. An assembly according to claim 12, characterised in that the first surface formation on the catch and the first surface formation on the hub are triangular.

16. An assembly according to claim 12, characterised in that the second surface formation on the hub is a rounded formation projecting inwardly of the hub.

17. An assembly according to claim 12, characterised in that the second formation on the catch has a lower height than the first formation on the catch.

18. An assembly according to claim 12, characterised in that the second formation on the catch is triangular with a forward face that projects substantially vertically of the catch and a rear face that is inclined.

19. An assembly according to claim 12, characterised in that the catch is formed from a wall of an end fitting at the machine end of the inner cannula.

20. An assembly according to claim 12, characterised in that the catch is substantially rectangular.

21. An assembly of a tracheostomy tube and an inner cannula extending within the tracheostomy tube, the inner cannula including a tubular shaft and a machine end region arranged to fit within a hub at the machine end of the tracheostomy tube, characterised in that the machine end region of the inner cannula has a resilient catch in the machine end region arranged to engage the inside of the hub at the machine end of the tracheostomy tube to retain the inner cannula in the tracheostomy tube against an axial force tending to withdraw the inner cannula, that the catch has two inclined projections spaced from one another along it, and that the inner surface of the hub has a recess shaped to receive one of the projections on the catch and a projection positioned to engage the other projection on the catch and thereby deflect the catch inwardly when the inner cannula is pulled rearwardly out of the tube.

22. An inner cannula for an assembly of a tracheostomy tube having the inner cannula extending within the tracheostomy tube, the inner cannula including a tubular shaft and a machine end region arranged to fit within a hub at the machine end of the tracheostomy tube, characterised in that the machine end region of the inner cannula has a resilient catch in the machine end region arranged to engage the inside of the hub at the machine end of the tracheostomy tube to retain the inner cannula in the tracheostomy tube against an axial force tending to withdraw the inner cannula, that the catch has two inclined projections spaced from one another along it, and that the inner surface of the hub has a recess shaped to receive one of the projections on the catch and a projection positioned to engage the other projection on the catch and thereby deflect the catch inwardly when the inner cannula is pulled rearwardly out of the tube.
